(19) United States
(12) Patent Application Publication
(10) Pub. No.: US 2017/0210781 A1
(43) Pub. Date: Jul. 27, 2017

(54) POLYPEPTIDES AND USES THEREOF

(71) Applicant: OXFORD UNIVERSITY INNOVATION LIMITED, Botley (GB)

(72) Inventors: MILES NUNN, Oxford (GB); SUSAN MARY LEA, Oxford (GB); MATTHIJS MIKLAS JORE, Oxford (GB)

(21) Appl. No.: 15/316,458

(22) PCT Filed: Jun. 5, 2015
(86) PCT No.: PCT/GB15/51654

§ 371 Date: Dec. 5, 2016

(30) Foreign Application Priority Data
Jun. 5, 2014 (GB) 1410031.7

(51) Int. Cl. C07K 14/435; A61K 35/64; A61K 48/00

(52) U.S. Cl. CPC C07K 14/43527; A61K 48/00; A61K 35/64; C12Y 304/21043; A61K 38/00

(57) ABSTRACT

The invention relates to polypeptides, and in particular to polypeptides that are capable of inhibiting the activity or activation of the complement system. It also relates to nucleic acids that encode the polypeptides and to uses of the polypeptides.

The complement system helps or “complements” the ability of antibodies and phagocytic cells to clear pathogens from an organism. It forms part of the innate immune system. Down-regulation of complement activation has been demonstrated to be effective in treating several disease indications in animal models and in ex vivo studies. The present invention provides novel polypeptides that can be used for the treatment of diseases or disorders that relate to inappropriate activation of one or more of the complement pathways.

### CROSS REFERENCE TO RELATED APPLICATIONS

This application is a national phase application based on PCT/GB15/51654 filed on Jun. 5, 2015, which claims priority to GB 1410031.7, filed on Jun. 5, 2014, each of which is incorporated herein by reference in its entirety as if fully set forth herein.

### FIELD OF THE DISCLOSURE

This invention relates to polypeptides, and in particular to polypeptides that are capable of inhibiting the activity or activation of the complement system. The present invention also relates to nucleic acids that encode the polypeptides and to uses of the polypeptides.

### DESCRIPTION

The complement system helps or “complements” the ability of antibodies and phagocytic cells to clear pathogens from an organism. It forms part of the innate immune system. The complement system consists of over 30 proteins found in the blood and on cell membranes, those in solution are generally synthesized by the liver, and normally circulate as inactive precursors (pro-proteins). When stimulated by one of several triggers, proteases in the system cleave specific proteins to initiate an amplifying cascade of further cleavages. The end-result of this activation cascade is massive amplification of the response and activation of the cell-killing membrane attack complex, recruitment of white blood cells and release of pro-inflammatory mediators. Complement proteins account for about 5% of the globulin fraction of blood serum and can serve as opsonins.

The complement system may be activated mainly by three different biochemical pathways: the classical complement pathway, the alternative complement pathway, and the lectin pathway. The non-specific protease dependent pathway may also play a more limited role in complement activation.

The classical complement pathway is typically activated by antigen-antibody complexes, pentraxins, or apoptotic cells binding to the complement system protein C1q. Activation of the classical complement pathway is involved in tissue damage resulting from deposition of autoantibodies and immune complexes, which may occur in autoimmune diseases such as systemic lupus erythematosus, myasthenia gravis and Goodpasture's syndrome. Classical pathway activation is responsible for tissue injury in hyperacute xenograft rejection triggered by the direct binding of preformed host antibodies to the graft endothelium. Inappropriate complement activation is also an important mediator of ischemia and reperfusion injury occurring, for example, in stroke and myocardial infarction and after major surgery.

The lectin complement pathway is typically activated by microbial saccharides via the mannose-binding lectin or by ficolin in response to pathogens.

The alternative pathway is typically activated by surfaces of pathogens that have neutral or positive charge characteristics and do not express or contain complement inhibitors.

Activation of the complement system has many protective functions in immunity, both as a first line defense mechanism against pathogens and as a facilitator of acquired immunity. On the other hand, complement activation is a major cause of tissue injury in many pathological conditions.

Down-regulation of complement activation has been demonstrated to be effective in treating several disease indications in animal models and in ex vivo studies, including systemic lupus erythematosus and glomerulonephritis, rheumatoid arthritis, cardiopulmonary bypass and hemodialysis, hyperacute rejection in organ transplantation, myocardial infarction, reperfusion injury, and adult respiratory distress syndrome. In addition, other inflammatory conditions and autoimmune/immune complex diseases are also closely associated with complement activation, including thermal injury, severe asthma, anaphylactic shock, bowel inflammation, urticaria, angioedema, vasculitis, multiple sclerosis, myasthenia gravis, membranoproliferative glomerulonephritis, Sjogren's syndrome, renal disease, sepsis, paroxysmal nocturnal hemoglobinuria, psoriasis, transplant rejection, cancer, stroke, age-related macular degeneration, atypical haemolytic uremic syndrome, Crohn's disease and Alzheimer's disease.

The first complement specific drug to obtain market approval was eculizumab, an antibody against the complement component C5. However, because there are a wide range of diseases related to complement activation there is an increasing need for treatments that can reduce or prevent activity or activation of the complement. It may also be valuable to find therapies that are able to block or reduce activity of only some of the pathways, for example, only one or two of the three activation pathways (classical pathway, lectin pathway or alternative pathway) rather than all three. It is also advantageous to provide treatments that can reduce or prevent activity at specific points in the activation pathways. Inhibitors that inhibit points earlier in the complement activation pathway, for example, may be less broad acting and therefore better able to target specific diseases.

It is the aim of the present invention to provide novel polypeptides that can be used for the treatment of diseases or disorders that relate to inappropriate activation of one or more of the complement pathways.

In a first aspect, the present invention provides an isolated polypeptide comprising or consisting of:
(a) the amino acid sequence of any one of SEQ ID NOs: 1, 2, 3, 4, 5, 6, 7, 8, 9, 10, 11 or 12;
(b) a variant amino acid sequence having at least 60% sequence identity to (a);
(c) an amino acid sequence having at least 70%, 75%, 80%, 90%, 95%, 98% or 99% sequence identity to (a); or
(d) an active fragment of (a), (b) or (c) that is at least 40, 50, 60, 65, 70 or 75 amino acids in length.

The isolated polypeptide may comprise or consist of:
(a) (e) the sequence set out in SEQ ID NO 12 13, 14, 15, 16, 17, 18 or 19; or
(b) (f) an active variant amino acid sequence having at least 70%, 75%, 80%, 90%, 95%, 98% or 99% sequence identity to (e)

The polypeptides of SEQ ID NOs: 9, 10 and 11 all comprise the sequence of SEQ ID NO: 1, SEQ ID NOs: 9 and 10 include signal sequences. SEQ ID NOs 10 and 11 contain 6 Histidine tag sequences.

The polypeptide of the present invention may be a polypeptide comprising or consisting of the amino acid sequence shown in SEQ ID NO: 1, 2, 3, 4, 5, 6, 7, 8, 9, 10, 11, 12, 13, 14, 15, 16, 17, 18 or 19 with a 6 Histidine tag at the 5′ or 3′ end or another purification tag at the 5′ or 3′ end.

The polypeptide may comprise the amino acid sequence of SEQ ID NO: 1, 2, 3, 4, 5, 6, 7, 8, 9, 10, 11, 12, 13, 14, 15, 16, 17, 18 or 19 with additional amino acids at one and/or both ends. For example, a purification tag or other sequence may be added at the N-terminal end and/or at the C-terminal end. The polypeptide may comprise or consist of a fusion protein wherein the fusion protein comprises a sequence according to (a), (b), (c) or (d) is fused to one or more further polypeptides. The further polypeptides may be, for example, one or more active or inactive domains of a protein, one or more active or inactive full-length proteins and/or one or more active or inactive protein fragments.

The polypeptide may comprise an active fragment of a polypeptide having the sequence of SEQ ID NO 1, 2, 3, 4, 5, 6, 7, 8, 9, 10, 11 or 12. The active fragment may comprise or consist of at least at least 40, at least 42, at least 50, at least 55, at least 60, at least 65, at least 70 or at least 75 contiguous amino acids of the sequence set out in SEQ ID NO: 1, 2, 3, 4, 5, 6, 7, 8, 9, 10, 11 or 12. An active fragment may have 42 amino acids and/or have the sequence set out in SEQ ID NO: 12, 13, 14, 15, 16, 17, 18 or 19.

An active fragment may have 70%, 80%, 90%, 95%, 98% or 99% sequence identity to the sequence set out in SEQ ID NO 12, 13, 14, 15, 16, 17, 18 or 19

The polypeptide may be a fragment or variant of 1, 2, 3, 4, 5, 6, 7, 8, 9, 10 or 11 comprising a sequence having 70%, 80%, 90%, 95%, 98%, 99% or 100% sequence identity to SEQ ID NO 12, 13, 14, 15, 16, 17, 18 or 19.

Preferably an active fragment according to the invention displays at least 50%, 60%, 70%, 80%, 90% or more of the activity, in relation to the complement system, of the polypeptide from which it is derived. That is, for example, the fragment has least 50% of the inhibitory activity with respect to complement activation as the polypeptide from which it is derived. An active fragment according to the invention may display at least 20%, at least 30%, at least 40%, at least 50%, at least 60%, at least 70%, at least 80% or at least 90% or more of the activity, in relation to the complement system in a particular tissue, of the polypeptide from which it is derived. An active fragment with lower activity, for example about 20%, 30%, 40% or 50% of the inhibitory activity with respect to complement activation in a particular tissue compared to the polypeptide from which it is derived if it is also specific or targeted to the particular tissue.

If the polypeptide of the invention includes a signal and/or tag sequence one or more of these may be removed in order to release the active form of the polypeptide.

The invention also provides for a variant amino acid sequence having at least 60% sequence identity to SEQ ID NO: 1, 2, 3, 4, 5, 6, 7, 8, 9, 10, 11 or 12. The polypeptide of SEQ ID NO 1, 9, 10 and 11 of the invention was first isolated from the tick Rhipicephalus appendiculatus, and the invention provides for variants thereof including homologues derived from other tick species which retain at least about 60% sequence identity with SEQ ID NO: 1, 2, 3, 4, 5, 6, 7, 8, 9, 10, 11 or 12. Homologues may include paralogues and orthologues of the sequence of SEQ ID NO: 1, 2, 3, 4, 5, 6, 7, 8, 9, 10, 11 or 12, including, for example polypeptides from other tick species including R. sanguineus, R. bursa, Amblyomma. americanum, Amblyomma. cajennense, Amblyomma. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, Argas. reflexus, Ornithodoros erraticus, O. moubata moubata, O. m. porcinus, and O. savignyi.

Amino acid identity may be calculated using any suitable algorithm. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. ScL USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The variant sequences typically differ by at least 1, 2, 3, 5, 10, 20, 30, 50 or more mutations (which may be substitutions, deletions or insertions of amino acids). For example, from 1 to 50, 2 to 40, 3 to 30 or 5 to 20 amino acid substitutions, deletions or insertions may be made. The substitutions are preferably conservative substitutions, for example according to the table below. Amino acids in the same block in the second column and preferably in the same line in the third column may be substituted for each other.

| | | |
|---|---|---|
| Aliphatic | Non-Polar | G A P |
| | | I L V |
| | Polar-uncharged | C S T M |
| | | N Q |
| | Polar-charged | D E |
| | | K R |
| Aromatic | | H F W Y |

The polypeptides of the invention may also be provided as a fusion protein comprising a polypeptide of the invention genetically or chemically fused to another peptide. The purpose of the other peptide may be any purpose, including, to aid detection, expression, separation or purification of the protein. Alternatively the protein may be fused to a peptide such as an Fc peptide to increase the circulating half life of the protein. Examples of other fusion partners include beta-galactosidase, glutathione-S-transferase, or luciferase.

The polypeptides of the invention may be chemically modified, e.g. post-translationally modified. For example, they may be glycosylated, pegylated, phosphorylated or comprise modified amino acid residues. They may be modified by the addition of histidine residues to assist their purification or by the addition of a signal sequence to promote insertion into the cell membrane. Such modified polypeptides fall within the scope of the term “polypeptide” used herein.

Polypeptides of the invention may be in a substantially isolated form. It will be understood that the polypeptide may be mixed with carriers or diluents which will not interfere with the intended purpose of the polypeptide and the polypeptide will still be regarded as substantially isolated. A polypeptide for use in the invention may also be in a substantially purified form, in which case it will generally comprise the polypeptide in a preparation in which more than 50%, e.g. more than 60%, 70%, 80%, 90%, 95% or 99%, by weight of the polypeptide in the preparation is a polypeptide of the invention.

Polypeptides of the invention may be made synthetically or be recombinantly produced. For example, a recombinant polypeptide may be produced by transfecting mammalian, fungal, bacterial or insect cells in culture with an expression vector comprising a nucleotide sequence encoding the polypeptide operably linked to suitable control sequences, culturing the cells, extracting and purifying the polypeptide of the invention produced by the cells. The amino acid sequence of polypeptides for use in the invention may be modified to include non-naturally occurring amino acids or to increase the stability of the compound. When the polypeptides are produced by synthetic means, such amino acids may be introduced during production. The polypeptides may also be modified following either synthetic or recombinant production.

Polypeptides of the invention may also be produced using D-amino acidsA number of side chain modifications are known in the art and may be made to the side chains of the polypeptides of the invention providing the activity of the polypeptide is retained.

Preferably a polypeptide, or composition, of the invention has the ability to inhibit or reduce the activity or activation of the complement system. By “inhibit” it is meant that the polypeptide is able to inhibit or reduce the activation or activity of one or more of the alternative, classical or lectin pathways of complement activation. Preferably the polypeptide, or composition of the invention, is able to inhibit the lectin and/or classical pathway of the complement system. Preferably the polypeptide or composition of the invention has less, or no significant, effect on the alternative pathway of complement activation. The ability of a polypeptide or composition to reduce the effect of a complement pathway may be determined by any standard haemolytic or other suitable assays known in the art, such as, for example, those described in the Examples and in Giclas et al (1994) or an an enzyme immunoassay for the qualitative determination of functional classical, MBL and/or alternative complement pathways in human serum such as the assay described in Seelen et al. Journal of Immunological Methods Volume 296, Issues 1-2, January 2005, Pages 187-198, which is also known as a Wieslab® assay.

Preferably, the presence of a polypeptide or composition of the invention reduces red blood cell lysis in a suitable assay or demonstrates reduction in complement activation in a suitable assay by at least 20% compared to the same assay in the absence of a polypeptide or composition of the present invention. A complement inhibitor molecule, polypeptide or composition of the present invention may, more preferably reduce complement activation in a suitable assay by at least 30%, 40%, 50%, 60%, 70%, 80%, 90%, 95%, 98% or 100%.

A suitable assay for complement activation may be an enzyme immunoassay for the qualitative determination of functional classical, MBL and/or alternative complement pathways in human serum for example as described in Seelen et al. Journal of Immunological Methods Volume 296, Issues 1-2, January 2005, Pages 187-198, Activity of the polypeptide may be tested by using any complement inhibition assay. The polypeptide may have complement pathway inhibiting activity when tested using a complement inhibition assay.

The polypeptide may reduce the total activity of the complement system by more than 10%, more than 20%, more than 30%, more than 40%, more than 50%, more than 60%, more than 70%, more than 80% more than 90%, more than 95%, more than 98%, more than 99% or the polypeptide may reduce the activity of the complement by 100%.

The polypeptide may reduce the total activity of the complement system in one or more specific tissues by more than 10%, more than 20%, more than 30%, more than 40%, more than 50%, more than 60%, more than 70%, more than 80% more than 90%, more than 95%, more than 98%, more than 99% or the polypeptide may reduce the activity of the complement system in one or more specific tissues by 100%.

The polypeptide or composition of the invention may act in the complement pathway by binding to C5 and preventing or reducing activation of C5. For example the polypeptide may bind to C5 and prevent or reduce activiation of C5 by a C5-convertase, for example C4b2a3b and/or C3bBbC3b and/or C3bBbC3bP.

The polypeptide or composition of the invention may act in the complement pathway to inhibit or reduce the activity of one or more of MBL, C1q, C1r, C1s, MASP-1, MASP-2, MASP-3, C1-complex (C1qr2s2), C2, C3, C3b, C4, C4b, C3-convertase (C4b2a or C3bBb or C3bBbP), C5-convertase (C4b2a3b or C3bBbC3b or C3bBbC3bP), Properdin, C5, C5b, C6, C7, C8 or C9.

The polypeptide or composition of the invention may act in the complement pathway to inhibit or reduce the activity or one or more of MBL, C1q, C1r, C1s, MASP-1, MASP-2, MASP-3, C1-complex (C1qr2s2), C2, C3, C3b, C4, C4b, C3-convertase (C4b2a or C3bBb or C3bBbP), C5-convertase (C4b2a3b or C3bBbC3b or C3bBbC3bP) or Properdin.

The polypeptide or composition of the invention may act in the complement pathway to inhibit or reduce the activity of one or more of MBL, C1q, C3, C3b, C4a, C4b, C4b2, C2, C2b and C4b2a, more preferably one or more of C2, C4b2 or C4b2a.

The polypeptide or composition of the invention may act in the complement pathway to inhibit or reduce the activity of one or more components but not reduce the activity of one or more of properdin, C6, C7, C8 or C9.

The polypeptide may bind to or interact with one or more factors selected from MBL, C1q, C1r, C1s, MASP-1, MASP-2, MASP-3, C1-complex (C1qr2s2), C2, C3, C3b, C4, C4b, C3-convertase (C4b2a or C3bBb or C3bBbP), C5-convertase (C4b2a3b or C3bBbC3b or C3bBbC3bP), Properdin, C5, C5b, C6, C7, C8 and C9 and reduce its activity. The activity of downstream factors in the complement pathway may also be reduced because of reduced activity of the upstream factor.

The polypeptide may bind to or interact with one or more factors selected from MBL, C1q, C1r, C1s, MASP-1, MASP-2, MASP-3, C1-complex (C1qr2s2), C2, C3, C3b, C4, C4b, C3-convertase (C4b2a or C3bBb or C3bBbP), C5-convertase (C4b2a3b or C3bBbC3b or C3bBbC3bP) or Properdin and reduce its activity. The activity of downstream factors in the complement pathway may also be reduced because of reduced activity of the upstream factor.

The polypeptide may bind to or interact with one or more factors selected from MBL, C1q, C3, C3b, C4a, C4b, C4b2, C2, C2b and C4b2a, more preferably one or more of C2, C4b2 or C4b2a and reduce its activity. The activity of downstream factors in the complement pathway may also be reduced because of reduced activity of the upstream factor.

The polypeptide or composition of the invention may act on one or more proteins involved in the lectin, alternative and/or classical complement pathways. Preferably the polypeptide or composition of the invention acts on one or more proteins active in the lectin, alternative and classical complement pathways. The polypeptide of the composition may act on the lectin and classical complement pathways to a greater extent than on the alternative complement pathway. The polypeptide may decrease the activity of the classical and lectin complement pathways more than it reduces the activity of the alternative pathway. This is advantageous because an appropriate dosing level of the polypeptide could ablate classical and lectin pathway activity completely but leave some of the alternative pathway activity at a level sufficient to tackle/prevent bacterial and other infections. The polypeptide or composition of the invention may not act on proteins involved in the alternative complement pathway.

According to a further aspect, the invention provides a polynucleotide encoding a polypeptide of the invention. The polynucleotide may be DNA or RNA.

The invention may further provide a vector, for example an expression vector, comprising a polynucleotide of the invention. Such expression vectors are routinely constructed in the art of molecular biology and may for example involve the use of plasmid DNA and appropriate initiators, promoters, enhancers and other elements, such as for example polyadenylation signals, which may be necessary and which are positioned in the correct orientation in order to allow for protein expression. The coding sequences may also be selected to provide a preferred codon usage suitable for the host organism to be used. Other suitable vectors would be apparent to persons skilled in the art.

Preferably, a polynucleotide for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with an origin of replication, optionally a promoter for the expression of the said polynucleotide and optionally a regulator of the promoter. The vector is typically adapted to be used in vivo. The vector may be a gene therapy vector, for example an adenovirus vector, a lentivirus vector or a CRISP-R vector.

Promoters and other expression regulation signals may be selected to be compatible with the host cell for which expression is designed. Mammalian promoters, such as [beta]-actin promoters, may be used. Tissue-specific promoters are especially preferred. Viral promoters may also be used, for example the Moloney murine leukaemia virus long terminal repeat (MMLV LTR), the Rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences. This will allow the introduction of the polynucleotides of the invention into the genome of eukaryotic cells by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention may further provide a host cell comprising the polynucleotide and/or the vector of the invention. The host cell may be a cell of the subject to be treated.

According to a further aspect the invention provides a composition comprising one or more isolated polypeptides of the invention. The composition of the present invention may be formulated for use in vitro. The composition of the present invention may be formulated for use in vivo, for example in humans. The composition of the present invention may be formulated for use in animals, for example, mammals, horses, cattle, pigs, sheep, goats, dogs, cats, rodents, fish, reptiles or birds. The composition of the present invention may be a pharmaceutical composition.

According to a yet further aspect the invention provides a pharmaceutical composition comprising one or more of (i) an isolated polypeptide of the invention; (ii) a polynucleotide of the invention; (iii) a vector or the invention; and (iv) a host cell of the invention. The pharmaceutical composition may comprise further ingredients, for example, one or more pharmaceutically acceptable excipient or carrier.

The pharmaceutical composition of the present invention may comprise one or more further active ingredients in addition to the one or more isolated polypeptides of the invention. The pharmaceutical composition may comprise one or more further active ingredients that modulate the immune system.

The polypeptide, polynucleotide or composition of the invention may be intended to be administered by enteral or parenteral routes such as via oral, buccal, anal, pulmonary, intravenous, subcutaneous, intra-arterial, intramuscular, intraperitoneal, intraarticular, topical, inhalation, intraocular or other appropriate administration routes.

The polypeptide, polynucleotide or composition of the invention may be intended to be administered in a variety of dosage forms. It may be administered orally (e.g. as tablets, troches, lozenges, aqueous or oily suspensions, dispersible powders or granules), parenterally, subcutaneously, intravenously, intramuscularly, intraocularly, intranasally, transdermally, topically or by infusion techniques.

Typically the polypeptide, polynucleotide or composition of the invention is formulated for use with a pharmaceutically acceptable carrier or diluent and this may be carried out using routine methods in the pharmaceutical art. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes. Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusions may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose. Pharmaceutical compositions suitable for delivery by needleless injection, for example, transdermally, may also be used. The compositions according to the invention may be presented in all dosage forms normally used for topical application, in particular in the form of aqueous, aqueous-alcoholic or, oily solutions, of dispersions of the lotion or serum type, of anhydrous or lipophilic gels, of emulsions of liquid or semi-solid consistency of the milk type, obtained by dispersing a fatty phase in an aqueous phase (O/W) or vice versa (W/O), or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods. They may also be used for the scalp in the form of aqueous, alcoholic or aqueous-alcoholic solutions, or in the form of creams, gels, emulsions or foams or alternatively in the form of aerosol compositions also containing a propellant agent under pressure.

The amounts of the different constituents of the compositions according to the invention are those traditionally used in the fields in question.

Preferably a therapeutically effective amount of a polypeptide, polynucleotide or composition of the invention is administered or intended to be administered. The dose may be determined according to various parameters, especially according to the polypeptide, polynucleotide or composition used; the age, weight and condition of the patient to be treated; the route of administration; and the required regimen. Again, a physician will be able to determine the required route of administration and dosage for any particular patient. A typical daily dose is from about 0.001 to 50 mg per kg, preferably from about 0.0 lmg/kg to 10 mg/kg of body weight, according to the activity of the polypeptide, the age, weight and conditions of the subject to be treated, the type and severity of the disease and the frequency and route of administration. Preferably, daily dosage levels are from 0.5 mg to 2 g. Lower dosages may be used for topical administration.

According to a further aspect, the invention provides a composition comprising a polypeptide of the invention; a polynucleotide of the invention; a vector of the invention; or a host cell of the invention; for use in medicine.

According to a further aspect, the invention provides a composition comprising a polypeptide of the invention; a polynucleotide of the invention; a vector of the invention; or a host cell of the invention; for use in the treatment of a disease or a condition mediated by complement.

The treatment may be therapeutic or prophylactic.

According to a another aspect, the invention provides a method for the treatment of a disease or a condition mediated by complement comprising administering to a subject in need thereof an effective amount of a polypeptide of the invention; a polynucleotide of the invention; a vector of the invention; or a host cell of the invention.

A disease or disorder mediated by complement which may treatable or preventable by the polypeptide, polynucleotide or composition of the present invention may be any disease or disorder that results from, or results in, activation, or increased activation, of the complement system in a subject. The polypeptide, polynucleotide, composition or pharmaceutical composition of the present invention may be useful in preventing activation of the complement system so that it is kept at a normal level in an individual who is at risk of abnormal activation of the complement system or any disease or disorder related to abnormal activation of the complement system. The polypeptide, polynucleotide, composition or pharmaceutical composition of the present invention may be particularly useful in preventing activation of the classical pathway, and/or the lectin pathway with a less significant effect on the alternative pathway. This may be useful in preventing acute rejection in organ transplantation; tissue damage resulting from deposition of autoantibodies and immune complexes, which may occur in autoimmune diseases such as systemic lupus erythematosus, myasthenia gravis and Goodpasture's syndrome; tissue injury in hyperacute xenograft rejection triggered by the direct binding of preformed host antibodies to the graft endothelium; ischemia and reperfusion injury occurring, for example, in stroke and myocardial infarction and after major surgery; anti-phospholipid syndrome and cold agglutinin disease; arthritis; neuromyelitis optica; thrombotic microangiopathies; Sjogren's Syndrome; psoriasis; bullous pemphigod and related skin disorders; cardiovascular pulmonary disease; and dense deposit disease while leaving some alternative pathway activity sufficient to reduce or prevent bacterial and other infections.

The polypeptide, polynucleotide, composition or pharmaceutical composition of the present invention may be useful in reducing the activity of the complement system in a subject that has an abnormally active complement system. The polypeptide, polynucleotide, composition or pharmaceutical composition of the present invention may be useful in reducing activation of the classical pathway, alternative and/or the lectin pathway. This may be useful in treatment of acute rejection in organ transplantation; tissue damage resulting from deposition of autoantibodies and immune complexes, which may occur in autoimmune diseases such as systemic lupus erythematosus, myasthenia gravis and Goodpasture's syndrome; tissue injury in hyperacute xenograft rejection triggered by the direct binding of preformed host antibodies to the graft endothelium; ischemia and reperfusion injury occurring, for example, in stroke and myocardial infarction and after major surgery; anti-phospholipid syndrome; cold agglutinin disease; arthritis; neuromyelitis optica; thrombotic microangiopathies; Sjogren's Syndrome; psoriasis; bullous pemphigod and related skin disorders; cardiovascular pulmonary disease; and dense deposit disease.

The polypeptide, polynucleotide or composition of the present invention may be for use in reducing activity of one or more of the complement pathways or inhibiting activation of one or more of the complement pathways.

The polypeptide, polynucleotide or composition of the present invention may be for in the treatment of a disease or disorder associated with increased activity in one or more of the complement pathways.

The polypeptide, polynucleotide or composition of the present invention may be for in the treatment of a disease or disorder associated with inappropriate activation of one or more the complement pathways.

A disease or disorder mediated by complement may include an inflammatory disease, ischemia, reperfusion injury, an autoimmune disease, an infection, an infection disease, transplant rejection, an ocular disease or a cancer.

A disease or disorder mediated by complement may also include a disease or disorder selected from systemic lupus erythematosus and glomerulonephritis, rheumatoid arthritis, cardiopulmonary bypass and hemodialysis, hyperacute rejection in organ transplantation, myocardial infarction, reperfusion injury, trauma, adult respiratory distress syndrome, thermal injury, asthma, anaphylactic shock, bowel inflammation, urticaria, angioedema, vasculitis, multiple sclerosis, myasthenia gravis, membranoproliferative glomerulonephritis, Sjogren's syndrome, renal disease, sepsis, paroxysmal nocturnal hemoglobinuria, psoriasis, transplant rejection, cancer, stroke, age-related macular degeneration, atypical haemolytic uremic syndrome, Crohn's disease and Alzheimer's disease.

Polypeptides and compositions of the present invention may be particularly useful in the treatment or prevention of acute rejection, nerve disorders mediated by antibody mediated complement activation (e.g. myasthenia gravis, Guillain-Barre syndrome, Miller-Fisher syndrome, neuromyelitis optica) and anti-phospholipid syndrome.

The present invention further provides a method of reducing the activity or activation of the classical complement pathway, alternative complement pathway and/or the lectin complement pathway in a subject, the method comprising the step of administering an effective amount of a polypeptide, polynucleotide or composition of the invention.

The polypeptide, polynucleotide or composition of the invention may be advantageous because a unit dose of the polypeptide can reduce the activity of the classical and/or lectin complement pathways, preferably both, to about 7 times greater extent than the alternative pathway. Therefore, the polypeptides, polynucleotides or compositions of the invention may be used to reduce the activity of the classical pathway and the lectin pathway without a significant effect on the alternative pathway. This aspect may be particularly useful in pathologies where risk of secondary infection is high (e.g. transplant or burns) or where pathology is caused by an infectious organism that is subject to control by the alternative pathway of complement activation. At a suitable dosage the activity of the classical and lectin complement pathways may be greatly reduced while the activity of the alternative pathway is only slightly reduced.

The present invention further provides a method of treating a disease or disorder associated with abnormal and/or increased activity of the complement pathway in a subject, wherein the method comprises administering to the subject an effective amount of a polypeptide, polynucleotide or composition of the invention.

The present invention further provides the use of a polypeptide, polynucleotide or composition of the invention in an in vitro method. For example, the polypeptide of the present invention may be used for investigating the complement pathways. The polypeptide of the present invention may be used to raise antibodies that specifically bind to the polypeptide of the invention. An isolated nucleic acid of the invention may be used as a polynucleotide probe in an in vitro method.

The polypeptide of the present invention or the polynucleotide of the present invention may be used in a diagnostic assay to test the activation of the complement system. For example the polypeptide or the isolated nucleic acid of the present invention may be used in a diagnostic assay to distinguish activation of the classical pathway or the lectin pathway from activation of the alternative pathway.

The present invention provides a method of providing a polypeptide of the invention, the method comprising expressing the polypeptide in suitable cell.

The cell may be a bacterial cell, a yeast cell, an insect cell or a mammalian cell. The cell may be a Drosophila S2 cell.

The polypeptide may be synthesised chemically in vitro.

The skilled man will appreciate that preferred features of any one embodiment and/or aspect and/or claim of the invention may be applied to all other embodiments and/or aspects and/or claims of the invention.

There now follows by way of example only a detailed description of the present invention with reference to the accompanying drawings, in which;

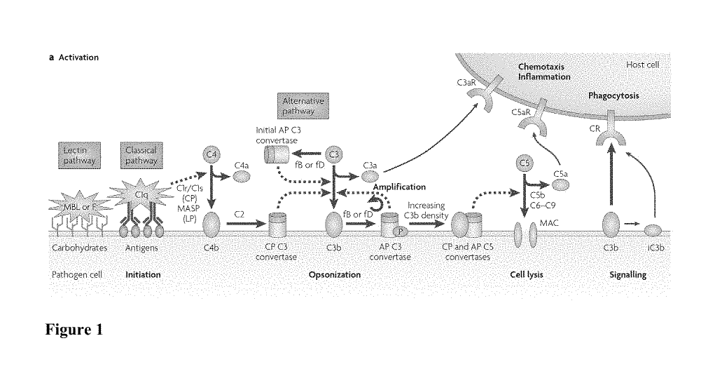

FIG. 1—shows an overview of the classical pathway, lectin pathway and alternative pathway of the complement system.

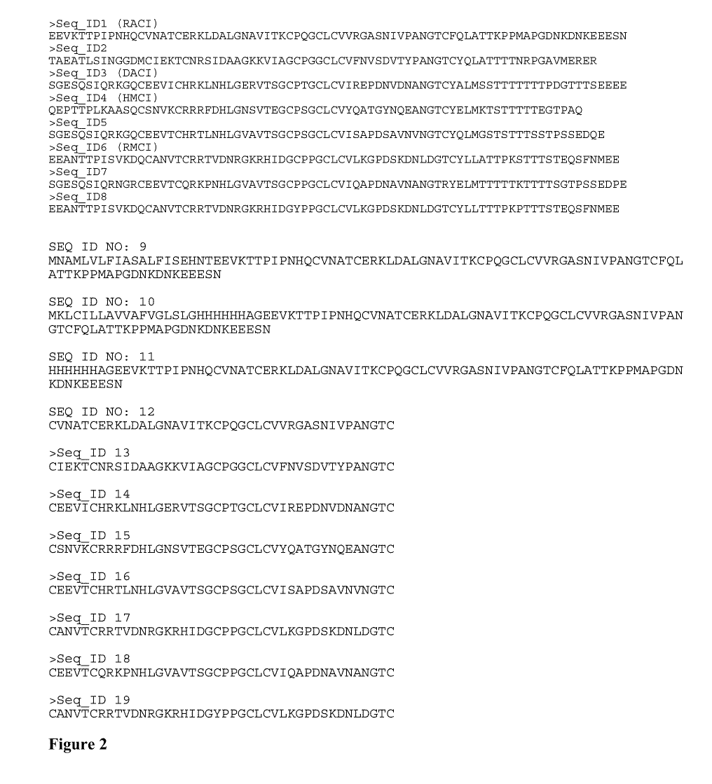

FIG. 2—shows the amino acid sequence of RaCI. SEQ ID NO 1 is also referred to herein as RaCI, it has no signal sequence or 6 His tag. SEQ ID NOs 2, 3, 4, 5, 6, 7 and 8 show homologues of SEQ ID NO: 1 (RaCI) and all have no signal sequence or 6 His tag. SEQ ID NO: 9 shows SEQ ID NO: 1 (RaCI) with the signal sequence underlined at the N-terminal end. SEQ ID NO: 10 shows SEQ ID NO: 1 (RaCI) with the signal sequence underlined followed by a 6His tag. SEQ ID NO; 11 shows SEQ ID NO: 1 (RaCI) with a 6 His tag and no signal sequence.

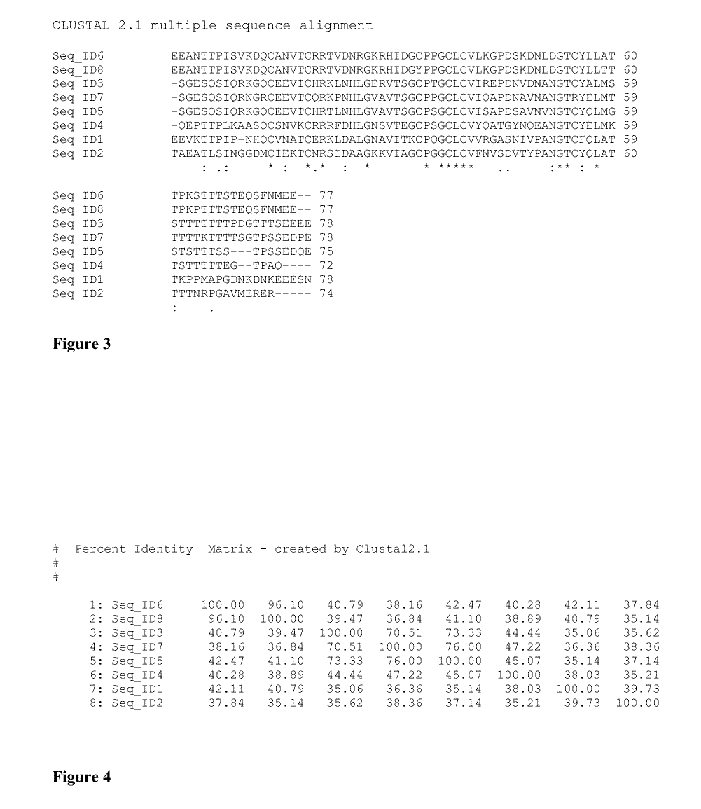

FIG. 3 shows a CLUSTAL 2.1 multiple sequence alignment of SEQ ID NOs 1 to 8.

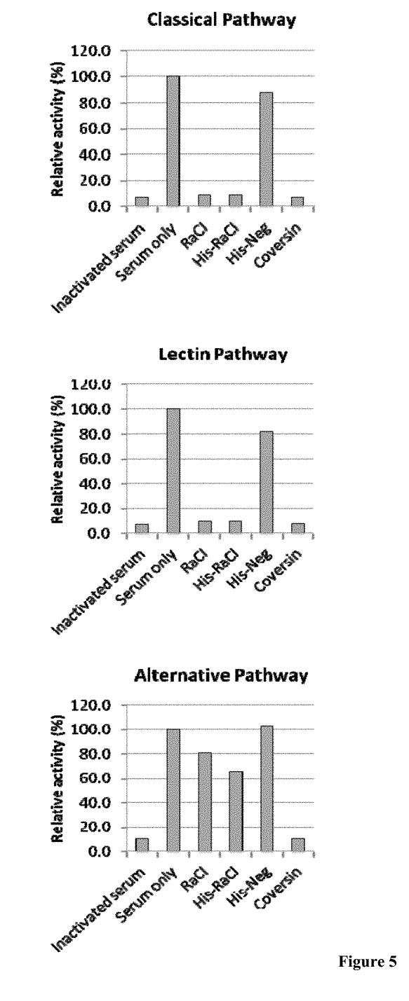

FIG. 4 shows a Percentage Identity Matrix—created by Clustal 2.1 of SEQ ID NOs 1 to 8.

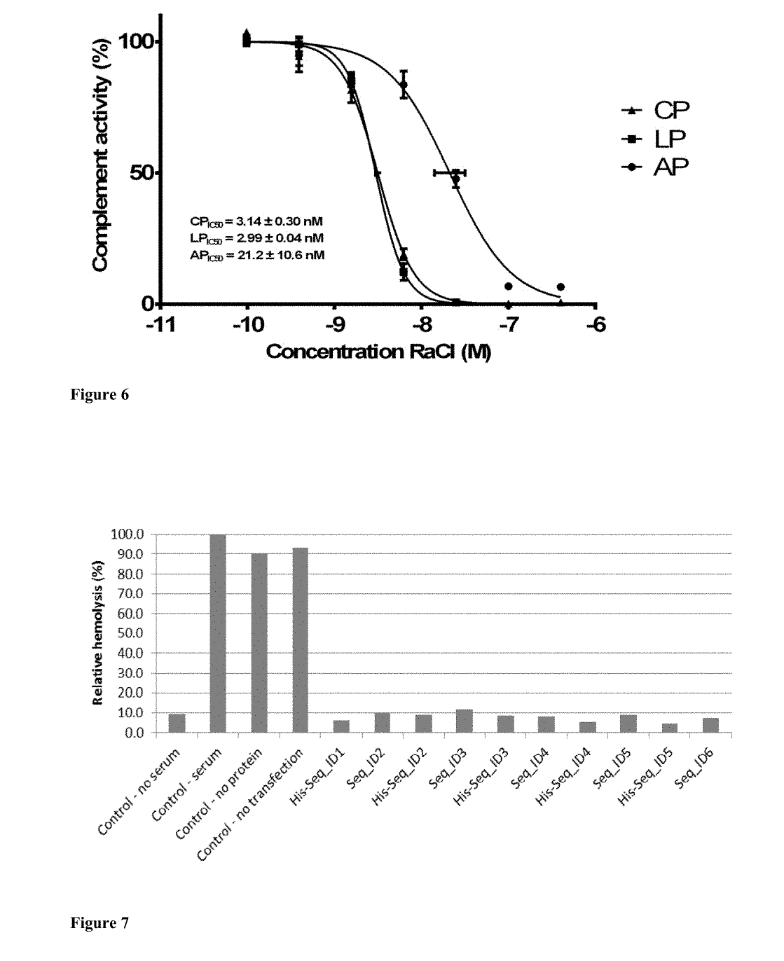

FIG. 5—shows the results of complement inhibition assays using the peptide of SEQ ID NO: 1 (labelled RaCI) and SEQ ID NO: 11 (labelled His-RaCI) for each of the classical pathway, the lectin pathway and the alternative pathway. The His-Neg polypeptide is a supernatant of a non-related His-tagged protein used as a negative control. 17 μg of purified Coversin (OmCI) is used as a positive control, this inhibits all the complement pathways as it inhibits the Terminal Pathway of the complement system. The activity of serum only is set to 100%. The values shown are the average of two repeats,

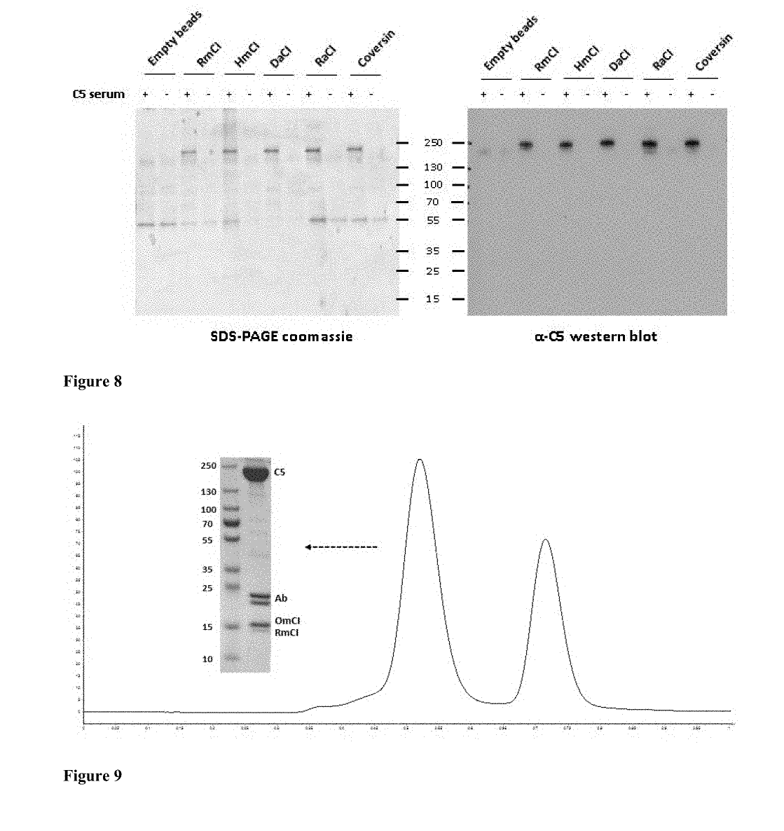

FIG. 6 shows effect of 6 His-RaCI on the Classical Pathway (CP), Lectin Pathway (LP) and Alternative Pathway (AP). The maximum and minimum values of each of the fitted curves were set to 100% and 0% activity respectively. Values are averages of three repeats and error bars indicate the standard error.

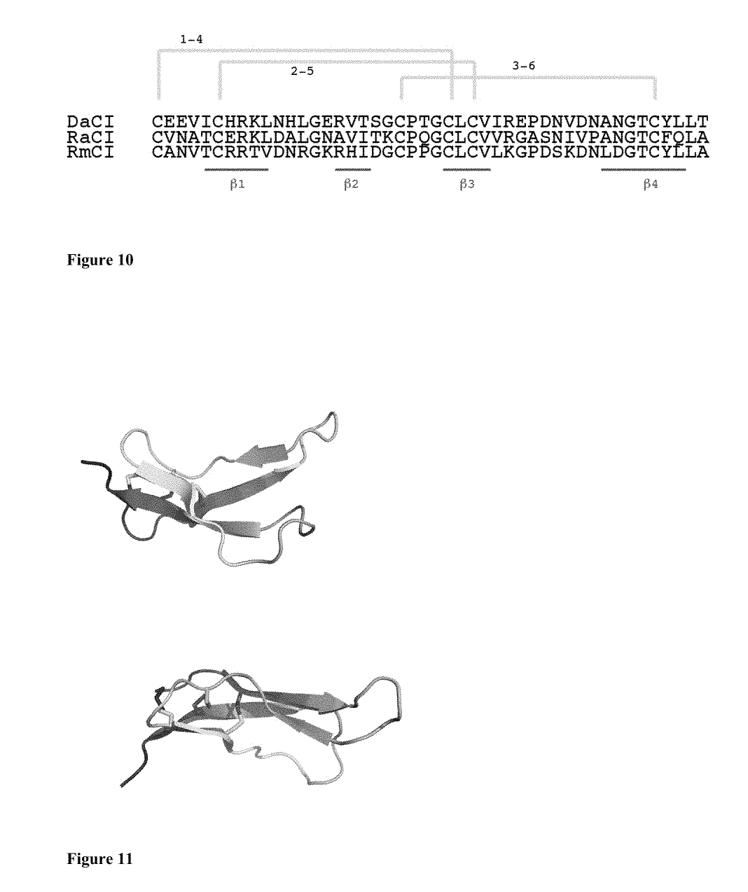

FIG. 7 shows complement inhibiting activity of RaCI and various of its homologues. Proteins corresponding to SEQ ID NOs 1-6 were tested. Some of the proteins were tested with the 6 His tag attached to the N-terminus as shown, e.g. “His-Seq_ID2” some of the proteins were tested without the 6 His tag, e.g “SEQ_ID2”. It was found that the 6 His tag did not significantly affect activity. Supernatants of stable S2 cell lines were checked for inhibiting activity in a hemolysis assay using sensitised sheep red blood cells, following the protocol described by Giclas P C, (National Jewish Center for Immunology and Respiratory Medicine, Denver, Colo., Publication Name: Current Protocols in Immunology, Unit Number: Unit 13.1, DOI: 10.1002/0471142735.im1301s09)

Normal human serum was used to a final dilution of 1/80,

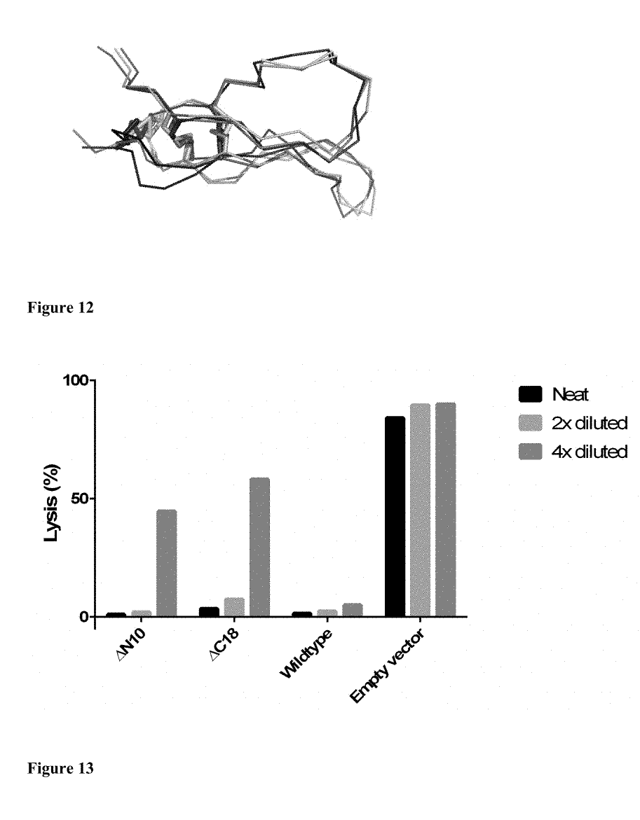

FIG. 8 shows the results of a pulldown assay of C5 by inhibitors of the present invention. Four inhibitors were covalently coupled to NHS-activated magnetic beads (Pierce Thermo Scientific). These all bound C5 from human serum as confirmed by western blot analysis with a poly-clonal anti-C5 antibody (CompTech, USA). C5 depleted serum indicated by (−) was used as a negative control. These inhibitors all work by binding C5 as evidenced by the fact that we can use inhibitors covalently coupled to beads to pull down human C5 from serum samples,

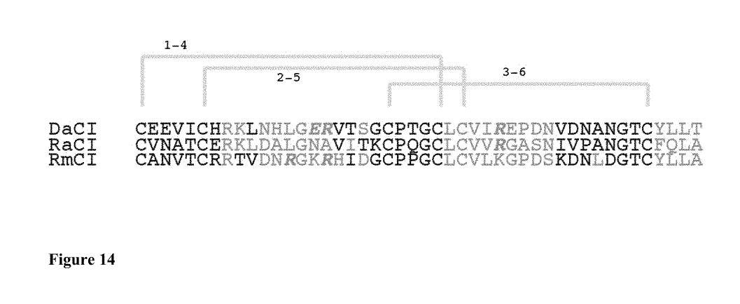

FIG. 9 shows a size exclusion elution profile and coomassie gel of the C5-OmCI-RmCI-Eculizumab complex. C5 was purified from human serum using His-tagged OmCI. Pure OmCI-C5 complexes were then mixed with threefold molar excess of Eculizumab (Ab) and RmCI. The insert gel shows the pooled fractions of the first peak. The second peak contains free antibody and RmCI. These inhibitors bind C5 in a different way to both the previous tick protein (OmCI/Coversin) or Eculizumab as evidenced by the fact we can form a quaternary complex of the four proteins,

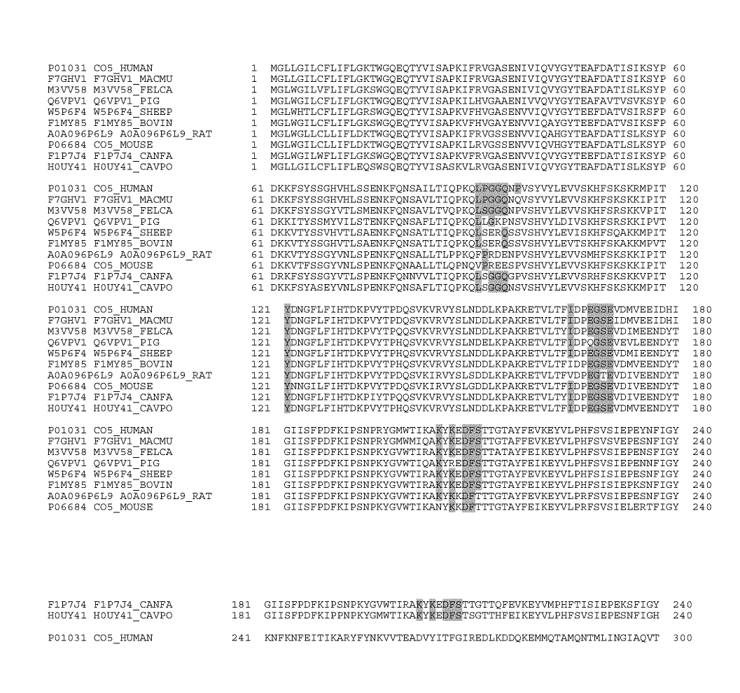

FIG. 10 shows Sequence alignment of the three inhibitors for which we have the structure. The pattern of disulphide bonds is indicated by the yellow lines and numbering above the alignment. The extent of secondary structure (as defined by DSSP) in the highest resolution structure (DaCI) is indicated below in blue. The full structure of three of these inhibitors that represent the level of sequence diversity seen in the family (less than 50% sequence identity within the folded core of the protein that is required for function),

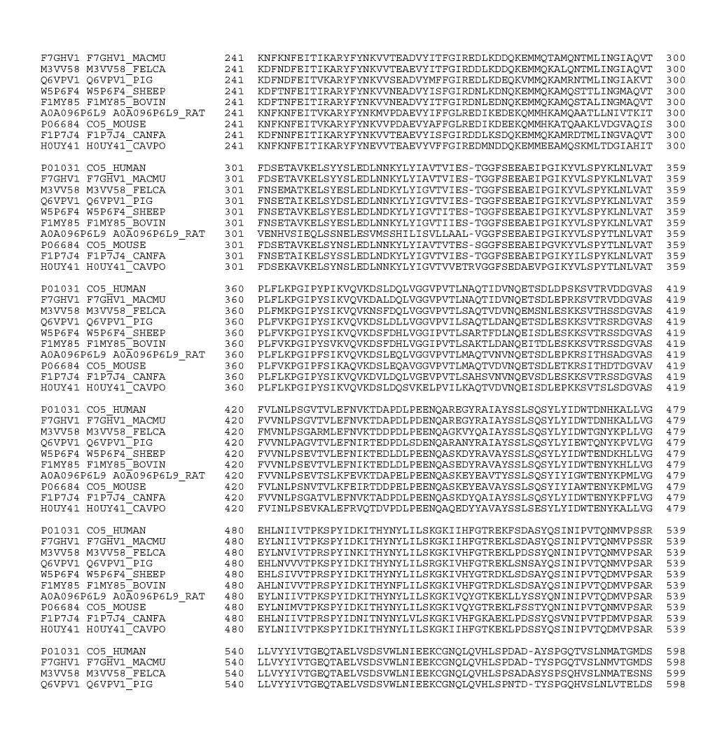

FIG. 11 shows cartoon representations of the DaCI structure (coloured Blue to Red from N-terminus to C-terminus) with the disulphides shown as sticks. The views are related by a 90 degree rotation,

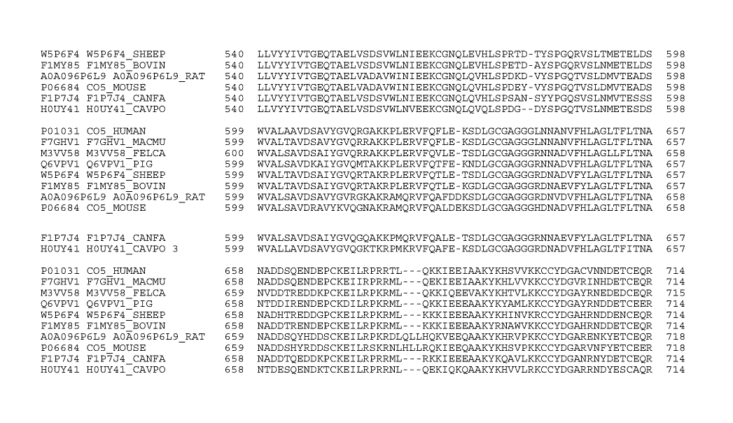

FIG. 12 shows overlay of the ribbon representations of the three inhibitors for which we have structure DaCI (coloured blue to red), RaCI (black) and RmCI (grey). The root mean square deviation between the backbone atoms for the structures is 1 Å 2. We have the full structures of each of these three inhibitors in complex with C5 to define exactly how the inhibitors interact with C5. This reveals that the only portion of the molecule needed for function are the 42 residues that lie between the first and last CYS in addition to revealing precisely which residues within this region are directly in contact with C5

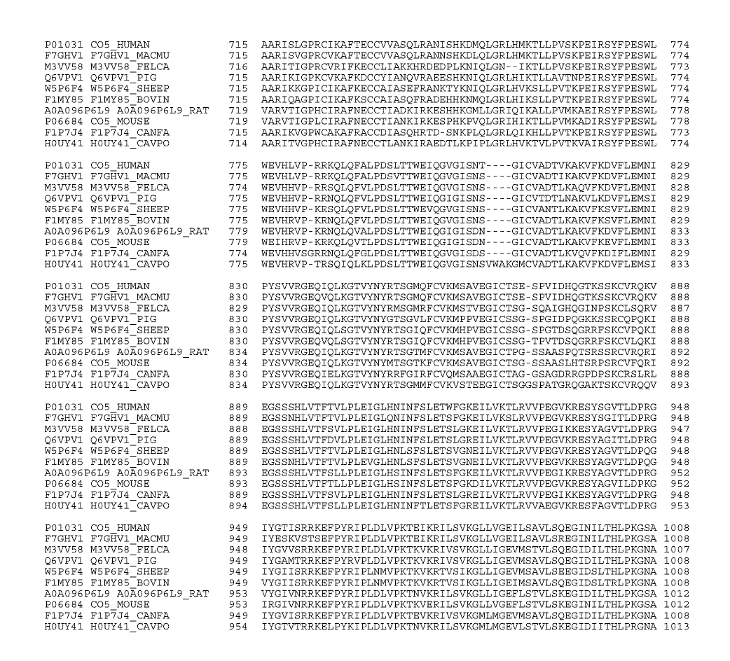

FIG. 13 shows complement inhibition by deletion mutants of RmCI. Mutants and wildtype RmCI were expressed in E. coli Shuffle T7 cells and cell lysates were tested for inhibitory activity in a haemolysis assay. Empty vector serves as a negative control. Values are means of two experimental replicates.

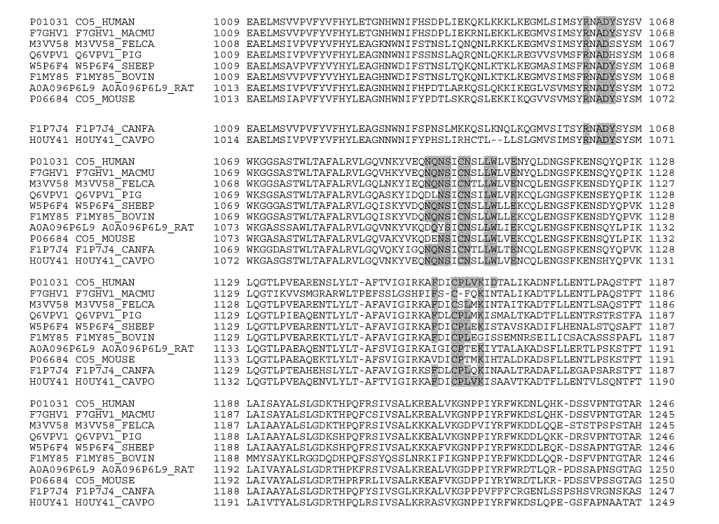

FIG. 14 shows a sequence alignment based on structures of the inhibitors bound to C5. Residues coloured green are buried in formation of the complex with C5. Those shown in green italics forms specific salt bridges with residues in C5.

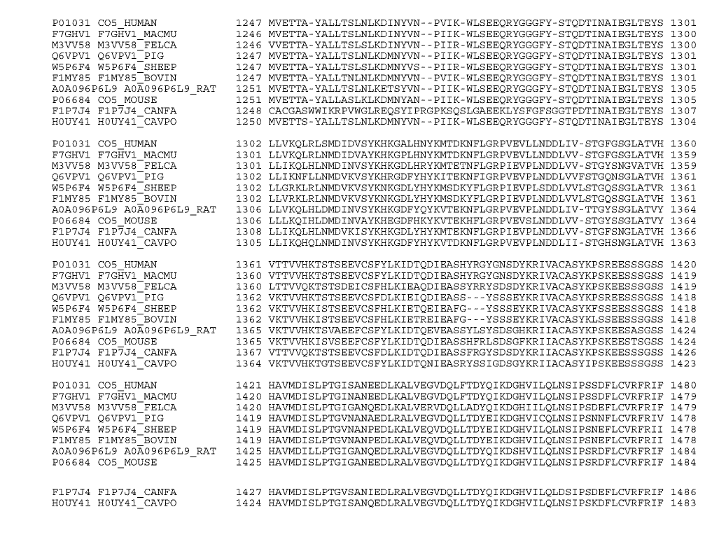

FIG. 15 shows Cross-species alignment of complement C5. Human C5 is the top sequence in each block and residues highlighted in blue are those that are buried in formation of the complex with DaCI/RaCI/RmCI. Highlighting in lower sequences shows which of the contact residues are conserved in other species. (We know that these inhibitors have full activity against human and guinea pig sera (top and bottom sequences in alignment) partial activity against pig sera (4th sequence from top) and no activity against rat and mouse (3rd and 4th sequences up from bottom.

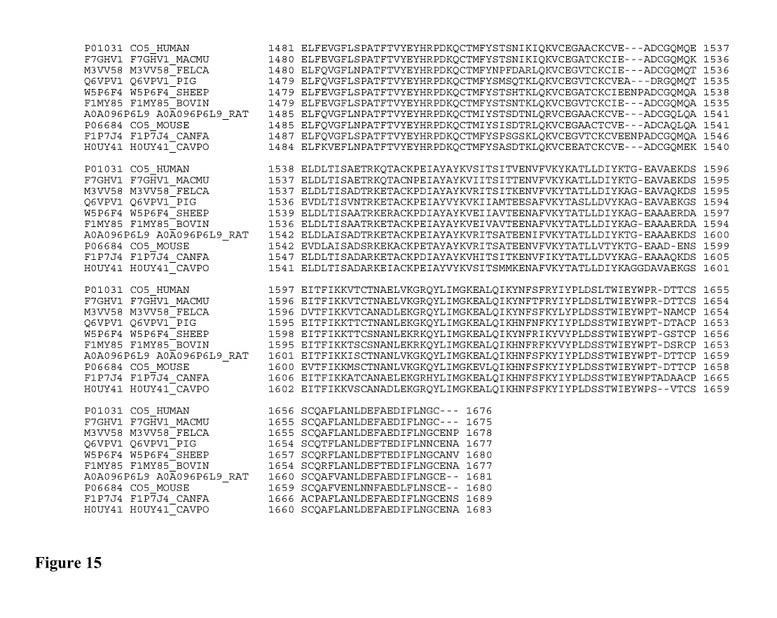

FIG. 16 shows cross-species activity of DaCI/RaCI/RmCI/HmCI tested in a classical pathway haemolysis assay. Final serum concentrations in the assay are 1/80 (human) and 1/640 (guinea pig). Values are averages of three repeats and error bars indicate the standard error, except for the mouse assay in which values are averages of two repeats. Values are normalised to PBS (100% activity) and empty wells (0%).

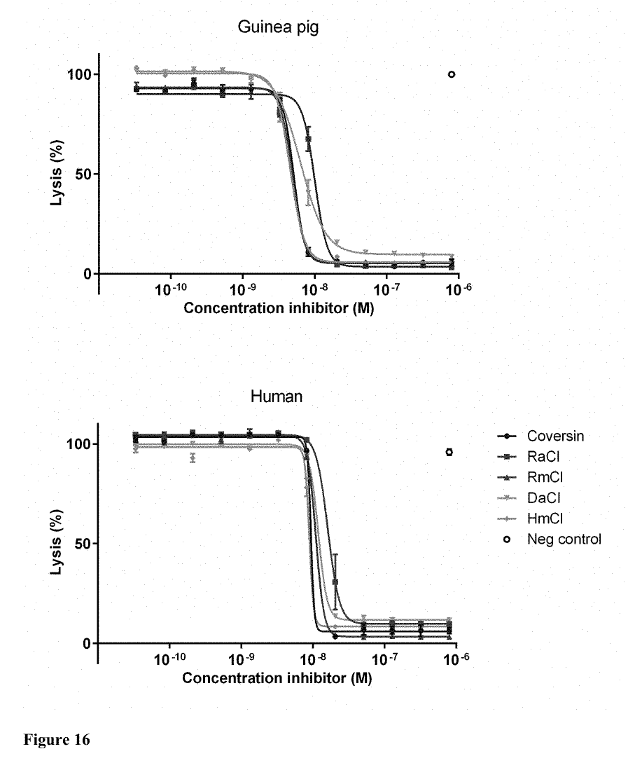

FIG. 17 shows picture of complex between DaCI (coloured blue at N-terminus to red at C-terminus) and complement C5 (wheat). The present inventors have developed a competition assay which reveals that these inhibitors do not act by stopping interaction with the C5 convertase (as has been the assumed mechanism for earlier C5 inhibitors) and our structure suggests the mechanism of inhibition is more likely to be that the inhibitors lock C5 into a conformation which is incompatible with activation. In terms of therapy this means that presence in the patient of inhibited C5 will further act to prevent activation of any un-inhibited C5 present by competing for binding to the activating enzyme,

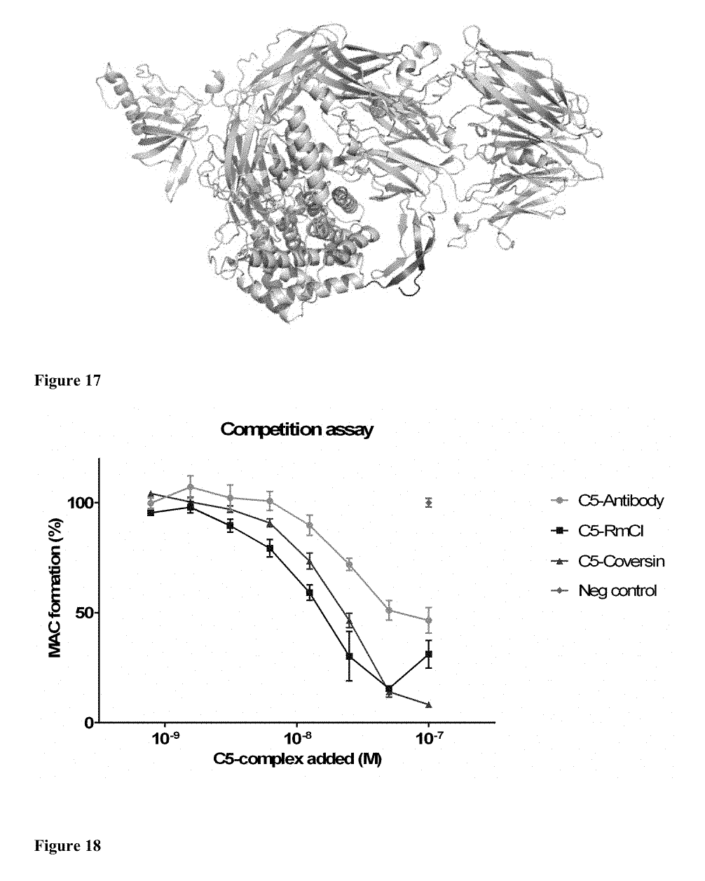

FIG. 18 shows Competition assay with C5-ligand complexes. Pure C5 (CompTech, USA) was mixed with two-fold molar excess ligand, and complexes were purified by a size exclusion chromatography step. Purified complexes were mixed with diluted serum in a Wieslab-based Classical pathway ELISA. Antibody is a Fab fragment based on the Eculizumab drug and Neg control is a tick protein not targeting Complement. Values are means of three repeats and error bars indicate the standard error.

Materials and Methods

Sequence and expression of polypeptides. Sequences of SEQ ID NO 1 to 11: were expressed from pExpres2-2 vectors in Drosophila S2 cells (ExpreS2ion Biotechnologies, Denmark). SEQ ID NO: 9 and SEQ ID NO: 10 are shown with the signal sequences underlined. SEQ ID NOs 1 to 8, 11 and 12 are shown without signal sequences. The signal sequences are cleaved during expression in Drosophila S2 cells to provide the peptides shown in SEQ ID NOs 1-8, 11 and 12. The signal sequences for the homologues SEQ ID NO 2 to 8 may be the same or similar to the signal sequence show underlined in SEQ ID NO: 9.

SEQ ID NOs: 9
(MNAMLVLFIASALFISEHNTEEVKTTPIPNHQCVNATCERKLDALGNAV
ITKCPQGCLCVVRGASNIVPANGTCFQLATTKPPMAPGDNKDNKEEESN)
and
SEQ ID NO: 10
(MKLCILLAVVAFVGLSLGHHHHHHAGEEVKTTPIPNHQCVNATCERKLD
ALGNAVITKCPQGCLCVVRGASNIVPANGTCFQLATTKPPMAPGDNKDNK
EEESN)

contain signal peptides for secretion (underlined), as predicted by SignalP 4.1 (http://www.cbs.dtu.dk/services/SignalP/). The proteins were transiently expressed for 72 hours according to the manufacturer's protocol (ExpreS2ion Biotechnologies, Denmark). Spent medium was cleared by centrifugation and tested for anti-complement activity using complement inhibition assays.

Purification of His6-RaCI. RaCI fused to an N-terminal His6-tag (His6-RaCI) was expressed from a pExpres2-2 vector in Drosophila S2 cells (ExpreS2ion Biotechnologies, Denmark). The generation of a stable cell line and expression of His6-RaCI were done according to the manufacturer's protocol (ExpreS2ion Biotechnologies, Denmark). Cell cultures were cleared by centrifugation and His6-RaCI was purified from the supernatant using a complete His-Tag Purification column (Roche), followed by a gel filtration step.

Complement inhibition assay for His-6-RaCI. Complement inhibiting activity of a dilution series of His6-RaCI was determined using the Complement System Screen WIESLAB (Euro Diagnostica, Sweden), according to the manufacturer's protocol with the following modifications. Normal human serum was used in all conditions. To test for complement inhibition 2 μl of purified His6-RaCI to 100 μl of diluted serum before the incubation step. The effect of His6-RaCI on each of the three pathways was tested with the different buffers and ELISA strips provided with the kit. The dilution series were used to calculate the IC50 values of His6-RaCI for each of the pathways.

The results of this assay are shown in FIG. 4. The effect of His6-RaCI on the Classical Pathway (CP), Lectin Pathway (LP) and Alternative Pathway (AP). The maximum and minimum values of each of the fitted curves were set to 100% and 0% activity respectively. Values are averages of three repeats and error bars indicate the standard error.

Complement inhibition assays for spent medium. Complement inhibiting activity of spent medium was determined using the Complement System Screen WIESLAB (Euro Diagnostica, Sweden), according to the manufacturer's protocol with the following modifications. Normal human serum was used in all conditions. To test for complement inhibition 5 μl of spent medium was added to 100 μl of diluted serum before the incubation step. The effect of SEQ ID NO: 1 on each of the three pathways was tested with the different buffers and ELISA strips provided with a Wieslab kit. The Wieslab kit is an ELISA kit having three different coatings on ELISA plates to activate each of the three pathways. Inhibitor is added to serum to before it is added to an ELISA well. % inhibition is proportional to amount of inhibitor added but should be up to 100%. The assay is performed as described in Seelen et al. Journal of Immunological Methods Volume 296, Issues 1-2, January 2005, Pages 187-198.

### RESULTS

The results of the complement inhibition assays for spent medium are shown in FIG. 3 the effect of SEQ ID NO: 1 on the Classical Pathway, Lectin Pathway and Alternative Pathway is shown. His-Neg is a supernatant of a non-related His-tagged protein used as a negative control. 17 ug of purified Coversin (OmCI) is used as a positive control, it inhibits all pathways since it inhibits the Terminal Pathway of the complement system. The activity of serum only is set to 100%. Values are averages of two repeats.

1. An isolated polypeptide comprising or consisting of:
(a) the amino acid sequence of any one of SEQ ID NOs: 1, 2, 3, 4, 5, 6, 7, 8, 9, 10, 11 or 12;
(b) a variant amino acid sequence having at least 60% sequence identity to (a);
(c) an amino acid sequence having at least 70%, 75%, 80%, 90%, 95%, 98% or 99% sequence identity to (a); or
(d) an active fragment of (a), (b) or (c) that is at least 40, 42, 50, 60, 65, 70 or 75 amino acids in length.

2. The isolated polypeptide according to claim 1 consisting of:
(a) the sequence set out in SEQ ID NO: 1, 2, 3, 4, 5, 6, 7, 8, 9, 10, 11 or 12;
(b) a variant amino acid sequence having at least 60% sequence identity to (a);
(c) an amino acid sequence having at least 70%, 75%, 80%, 90%, 95%, 98% or 99% sequence identity to (a); or
(d) an active fragment of (a), (b) or (c) that is at least 40, 42, 50, 60, 65, 70 or 75 amino acids in length.

3. An isolated polypeptide according to claim 1 or claim 2 comprising or consisting of:
(e) the sequence set out in SEQ ID NO 12, 13, 14, 15, 16, 17, 18 or 19; or
(f) an active variant amino acid sequence having at least 70%, 75%, 80%, 90%, 95%, 98% or 99% sequence identity to (e)

4. The isolated polypeptide according to any one of claims 1 to 3 comprising the amino acid sequence of SEQ ID NO: 1, 2, 3, 4, 5, 6, 7, 8, 9, 10, 11 12, 13, 14, 15, 16, 17, 18 or 19 with additional amino acids at one and/or both ends.

5. The isolated polypeptide according to any one of the preceding wherein the polypeptide consists of a fusion protein comprising a sequence according to (a), (b), (c) (d), (e) or (f) fused to one or more further polypeptides at the N- and/or C-terminal end.

6. The isolated polypeptide according to any one of the preceding claims wherein the polypeptide reduces the activity of the complement pathway or inhibits activation of the complement pathway.

7. The isolated polypeptide according to any one of the preceding claims wherein the polypeptide reduces the activity of or inhibits activation of the classical complement pathway, alternative complement pathway and/or the lectin mediated complement pathway.

8. The isolated polypeptide according to any one of the preceding claims wherein the polypeptide reduces the activity of or inhibits the activation of the classical complement pathway and/or lectin mediated complement pathway to a greater extent than it reduces the activity of or inhibits the activation of the alternative complement pathway.

9. The isolated polypeptide according to any one of the preceding claims wherein the polypeptide reduces complement activation in a suitable assay by at least 30%, 40%, 50%, 60%, 70%, 80%, 90%, 95%, 98% or 100%.

10. The isolated polypeptide according to any one of the preceding claims wherein the polypeptide reduces the total activity of the complement system in one or more specific tissues by more than 10%, more than 20%, more than 30%, more than 40%, more than 50%, more than 60%, more than 70%, more than 80% more than 90%, more than 95%, more than 98%, more than 99% or the polypeptide may reduce the activity of the complement system in one or more specific tissues by 100%.

11. The isolated polypeptide according to any one of the preceding claims wherein the polypeptide binds to C5 and inhibits activation of C5, for example by a C5 convertase.

12. A polynucleotide encoding a polypeptide of the invention.

13. An expression vector, comprising a polynucleotide according to claim 12, for example a gene therapy vector.

14. A host cell comprising the polynucleotide according to claim 12 and/or the vector according to claim 13.

15. A composition comprising one or more isolated polypeptides according to any one of claims 1 to 11.

16. A pharmaceutical composition comprising one or more of (i) an isolated polypeptide according to any one of claims 1 to 11; (ii) a polynucleotide according to claim 12; (iii) a vector according to claim 13; and (iv) a host cell according to claim 14. and optionally the pharmaceutical composition may further comprise further ingredients, for example, one or more pharmaceutically acceptable excipient or carrier.

17. The pharmaceutical composition according to claim 16 further comprising one or more further active ingredients.

18. A composition comprising an isolated polypeptide according to any one of claims 1 to 11; a polynucleotide according to claim 12; a vector according to claim 13; a host cell according to claim 14; or a pharmaceutical composition according to claim 16 or claim 17, for use in medicine.

19. A composition comprising an isolated polypeptide according to any one of claims 1 to 11; a polynucleotide according to claim 12; a vector according to claim 13; a host cell according to claim 14; or a pharmaceutical composition according to claim 16 or claim 17 for use in reducing activity of the complement pathway or inhibiting activation of the complement pathway.

20. A composition comprising an isolated polypeptide according to any one of claims 1 to 11 a polynucleotide according to claim 12; a vector according to claim 13; a host cell according to claim 14; or a pharmaceutical composition according to claim 16 or claim 17 for use in the prevention or treatment of a disease or disorder associated with increased activity in the complement pathway.

21. A composition comprising an isolated polypeptide according to any one of claims 1 to 11; a polynucleotide according to claim 12; a vector according to claim 13; a host cell according to claim 14; or a pharmaceutical composition according to claim 16 or claim 17, for use in the prophylactic or therapeutic treatment of a disease or a condition mediated by complement.

22. A composition comprising an isolated polypeptide according to any one of claims 1 to 11; a polynucleotide according to claim 12; a vector according to claim 13; a host cell according to claim 14; or a pharmaceutical composition according to claim 16 or claim 17, for use in the prophylactic or therapeutic treatment of acute rejection in organ transplantation; tissue damage resulting from deposition of autoantibodies and immune complexes, which may occur in autoimmune diseases such as systemic lupus erythematosus, myasthenia gravis and Goodpasture's syndrome; tissue injury in hyperacute xenograft rejection triggered by the direct binding of preformed host antibodies to the graft endothelium; ischemia and reperfusion injury occurring, for example, in stroke and myocardial infarction and after major surgery; anti-phospholipid syndrome and cold agglutinin disease; arthritis; neuromyelitis optica; thrombotic microangiopathies; Sjogren's Syndrome; psoriasis; bullous pemphigod and related skin disorders; cardiovascular pulmonary disease; or dense deposit disease.

23. A composition comprising an isolated polypeptide according to any one of claims 1 to 11; a polynucleotide according to claim 12; a vector according to claim 13; a host cell according to claim 14; or a pharmaceutical composition according to claim 16 or claim 17, for use in the prophylactic or therapeutic treatment of an inflammatory disease, ischemia, reperfusion injury, an autoimmune disease, an infection, an infection disease, transplant rejection, an ocular disease, a cancer, systemic lupus erythematosus, glomerulonephritis, rheumatoid arthritis, complications of cardiopulmonary bypass and hemodialysis, hyperacute rejection in organ transplantation, myocardial infarction, reperfusion injury, trauma, adult respiratory distress syndrome, thermal injury, asthma, anaphylactic shock, bowel inflammation, urticaria, angioedema, vasculitis, multiple sclerosis, myasthenia gravis, membranoproliferative glomerulonephritis, Sjogren's syndrome, renal disease, sepsis, paroxysmal nocturnal hemoglobinuria, psoriasis, transplant rejection, cancer, stroke, age-related macular degeneration, atypical haemolytic uremic syndrome, Crohn's disease and Alzheimer's disease, nerve disorders mediated by antibody mediated complement activation (e.g. myasthenia gravis, Guillain-Barre syndrome, Miller-Fisher syndrome, neuromyelitis optica) and anti-phospholipid syndrome.

24. Use of a polypeptide, polynucleotide or composition of the invention in an in vitro method.

25. Use of a polypeptide of the present invention or a polynucleotide of the present invention in a diagnostic assay to test the activation of the complement system.

26. A method of treating a disease or disorder in a subject associated with abnormal increased activity of the complement pathway wherein the method comprises administering to the subject an effective amount of a composition comprising an isolated polypeptide according to any one of claims 1 to 11; a polynucleotide according to claim 12; a vector according to claim 13; a host cell according to claim 14; or a pharmaceutical composition according to claim 16 or claim 17.

27. A method of providing a polypeptide according to any one of claims 1 to 11 comprising expressing the polypeptide in suitable cell.

28. A method for providing a polypeptide according to any one of claims 1 to 11 comprising expressing the polypeptide in a Drosophila S2 cell.

29. A polypeptide, composition, pharmaceutical composition or method as described herein with reference to the figures and examples.
